(19) United States
(12) Patent Application Publication
(10) Pub. No.: US 2018/0064318 A1
(43) Pub. Date: Mar. 8, 2018

(54) ENDOSCOPE

(71) Applicant: FUJIFILM Corporation, Tokyo (JP)

(72) Inventor: Ryo KITANO, Kanagawa (JP)

(73) Assignee: FUJIFILM Corporation, Tokyo (JP)

(21) Appl. No.: 15/666,582

(22) Filed: Aug. 2, 2017

(30) Foreign Application Priority Data
Sep. 8, 2016 (JP) 2016-175852

(51) Int. Cl. A61B 1/05; A61B 1/00; A61B 1/005; A61B 1/01; A61B 1/045; A61B 1/06

(52) U.S. Cl. CPC A61B 1/051; A61B 1/00126; A61B 1/00009; A61B 1/0057; A61B 1/0661; A61B 1/01; A61B 1/045; A61B 1/05; A61B 1/06; A61B 1/00096

(57) ABSTRACT

An endoscope includes an imaging device that is provided in a tip portion of an insertion part. The imaging device includes an image-sensor that an image receiving surface thereof is disposed crossing a longitudinal direction of the insertion part, a substrate folded once or more that includes a sensor connecting portion which faces a surface of the image-sensor on which connection terminals are provided and is connected to the connection terminals and a wire connecting portion which extends from an outer end of the sensor connecting portion toward a base end side of the insertion part and a center of the image receiving surface, and a composite wire of which a plurality of cables connected to a cable connection surface of the wire connecting portion facing the base end of the insertion part are bundled. The composite wire includes a first shield conductor provided around the plurality of cables that are bundled.

### CROSS-REFERENCE TO RELATED APPLICATIONS

The present application claims priority under 35 U.S.C. § 119 to Japanese Patent Application No. 2016-175852, filed on Sep. 8, 2016. Each of the above application(s) is hereby expressly incorporated by reference, in its entirety, into the present application.

### BACKGROUND OF THE INVENTION

### 1. Field of the Invention

The present invention relates to an endoscope.

### 2. Description of the Related Art

An imaging device, which is mounted in a tip of an insertion part of an endoscope, generally includes an image sensor and a circuit substrate on which the image sensor is mounted, and a cable inserted into the insertion part is connected to the circuit substrate.

In an imaging unit disclosed in JP4916595B, a solid-state imaging element and an electronic component are mounted on a substrate and conducting wire portions of a plurality of signal wires inserted into a cable are connected to a predetermined region of the substrate. Further, in an imaging device disclosed in JP2008-118568A, a solid-state imaging element is mounted on one end of a flexible substrate, and a plurality of signal wires are connected to the other end, which is folded, of the flexible substrate. An electronic component is mounted on the flexible substrate between one end and the other end of the flexible substrate.

### SUMMARY OF THE INVENTION

Since the diameter of the insertion part of the endoscope to be inserted into a subject needs to be reduced, contents of the insertion part are densely disposed. However, since heat generated from the solid-state imaging element and the like is accumulated in a small space in which the contents of the insertion part are densely disposed, a heat dissipation path needs to be prepared in the small space. In this case, since it is not preferable that heat is dissipated to the tip side of the insertion part in a state in which the insertion part is inserted into the subject, it is preferable that the heat dissipation path is provided on the base end side of the insertion part. It is preferable that the heat dissipation path is ensured without inhibiting a reduction in the diameter of the insertion part.

The invention has been made in consideration of the above-mentioned circumstances, and an object of the invention is to provide an endoscope that can ensure a heat dissipation path having a more sufficient capacity toward the base end side of an insertion part.

An endoscope according to an aspect of the invention comprises an imaging device that is provided in a tip portion of an insertion part capable of being inserted into a body cavity. The imaging device includes: a solid-state imaging element that is disposed so that an image receiving surface of the solid-state imaging element crosses a longitudinal direction of the insertion part; a substrate that includes a first region portion which faces a surface of the solid-state imaging element on which connection terminals are provided and is connected to the connection terminals and a second region portion which extends from an outer end of the first region portion toward a base end side of the insertion part and a center of the image receiving surface, and is folded once or more; and a composite wire which extends from the base end side of the insertion part toward a tip side of the insertion part and of which a plurality of cables electrically connected to one surface of the second region portion facing a base end of the insertion part are bundled. The composite wire includes a first external conductor provided around the plurality of cables that are bundled, and the first external conductor is connected to a cable connection surface that is one surface of the second region portion to which the plurality of cables are connected.

According to the invention, it is possible to provide an endoscope that can ensure a heat dissipation path having a more sufficient capacity toward the base end side of an insertion part.

### BRIEF DESCRIPTION OF THE DRAWINGS

FIG. 1 is a diagram showing the structure of an endoscope system including an endoscope according to the invention.

FIG. 2 is a perspective view of an imaging device of a first embodiment.

FIG. 3 is a side view of the imaging device of the first embodiment.

FIG. 4 is a cross-sectional view of a transmission cable.

FIG. 5 is a perspective view of an imaging device of a second embodiment.

FIG. 6 is a perspective view of an imaging device of a third embodiment.

FIG. 7 is a partial cross-sectional side view of the imaging device of the third embodiment.

FIG. 8 is a partial cross-sectional side view of an imaging device of a fourth embodiment.

FIG. 9 is a side view of an imaging device of a fifth embodiment.

FIG. 10 is a side view of an imaging device of a sixth embodiment.

### DESCRIPTION OF THE PREFERRED EMBODIMENTS

Embodiments of the invention will be described below with reference to the drawings.

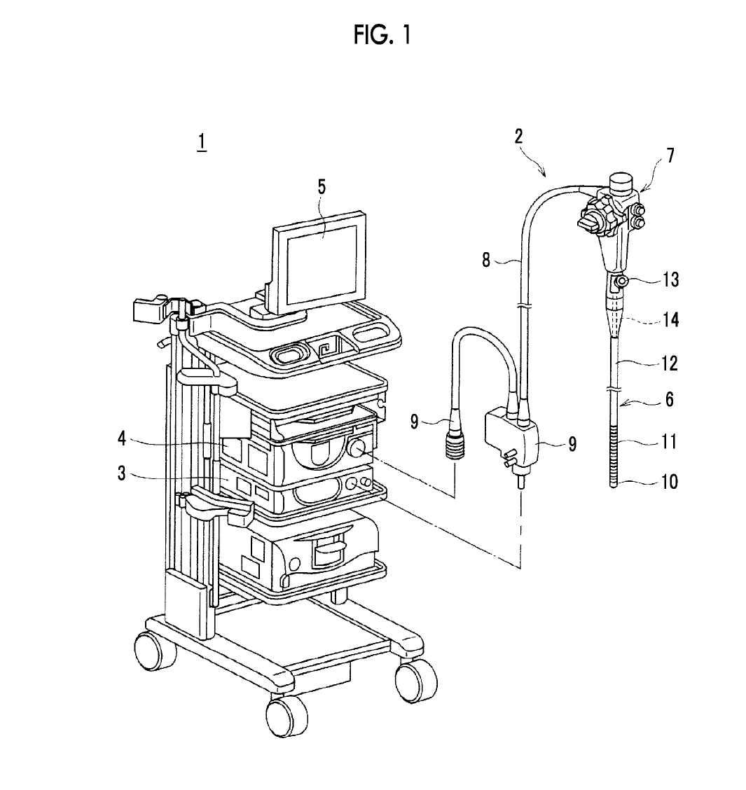

FIG. 1 shows an example of an endoscope system that is used to illustrate the embodiments of the invention.

The endoscope system 1 includes an endoscope 2, a light source unit 3, and a processor unit 4. The endoscope 2 includes an insertion part 6 that is capable of inserted into a subject, an operation unit 7 that is connected to the insertion part 6, and a universal cord 8 that extends from the operation unit 7. The insertion part 6 is provided on a tip side of the endoscope 2, and the universal cord 8 is provided on a base end side of the endoscope 2. The insertion part 6 includes a tip portion 10, a bendable portion 11 that is connected to the tip portion 10, and a soft portion 12 that connects the bendable portion 11 to the operation unit 7. The tip portion 10 is provided on a tip side on a longitudinal axis of the insertion part 6, and the soft portion 12 is provided on a base end side on the longitudinal axis. The tip side on the longitudinal axis of the insertion part 6 is synonymous with the tip side of the endoscope 2, and the base end side on the longitudinal axis of the insertion part 6 is synonymous with the base end side of the endoscope 2.

An illumination optical system that emits illumination light for illuminating a portion to be observed, an imaging device and an imaging optical system that image the portion to be observed, and the like are provided at the tip portion 10. The bendable portion 11 is adapted to be bendable in a direction orthogonal to the longitudinal axis of the insertion part 6, and an operation for bending the bendable portion 11 is manipulated by the operation unit 7. Further, the soft portion 12 is relatively flexible so as to be deformable according to the shape of the insertion path of the insertion part 6.

The operation unit 7 is provided with a button that is used to manipulate an imaging operation of the imaging device provided at the tip portion 10, a knob that is used to manipulate the operation for bending the bendable portion 11, and the like. Further, the operation unit 7 is provided with an inlet 13 into which a treatment tool, such as an electric scalpel, is introduced, and a treatment tool channel 14, which reaches the tip portion 10 from the inlet 13 and into which the treatment tool is inserted, is provided in the insertion part 6.

A connector 9 is provided at the end of the universal cord 8, and the endoscope 2 is connected to the light source unit 3, which generates illumination light to be emitted from the illumination optical system provided at the tip portion 10, and the processor unit 4, which processes a video signal acquired by the imaging device provided at the tip portion 10, through the connector 9. The processor unit 4 generates the video data of the portion to be observed by processing the input video signal, displays the generated video data on a monitor 5, and records the generated video data.

A light guide and a wire group are housed in the insertion part 6, the operation unit 7, and the universal cord 8. The illumination light, which is generated by the light source unit 3, is guided to the illumination optical system, which is provided at the tip portion 10, through the light guide, and a signal and power are transmitted between the imaging device, which is provided at the tip portion 10, and the processor unit 4 through the wire group.

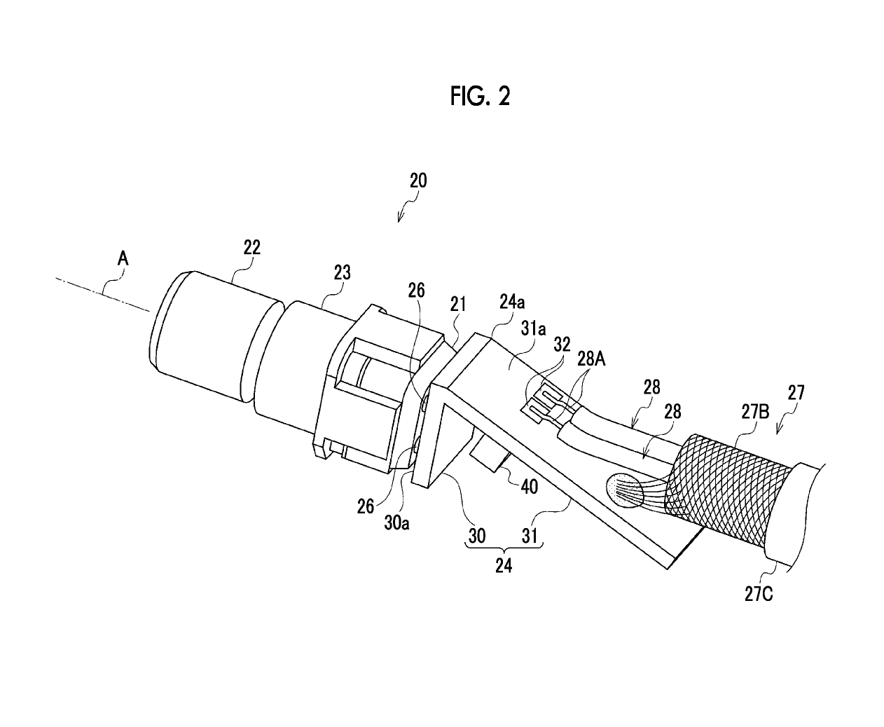

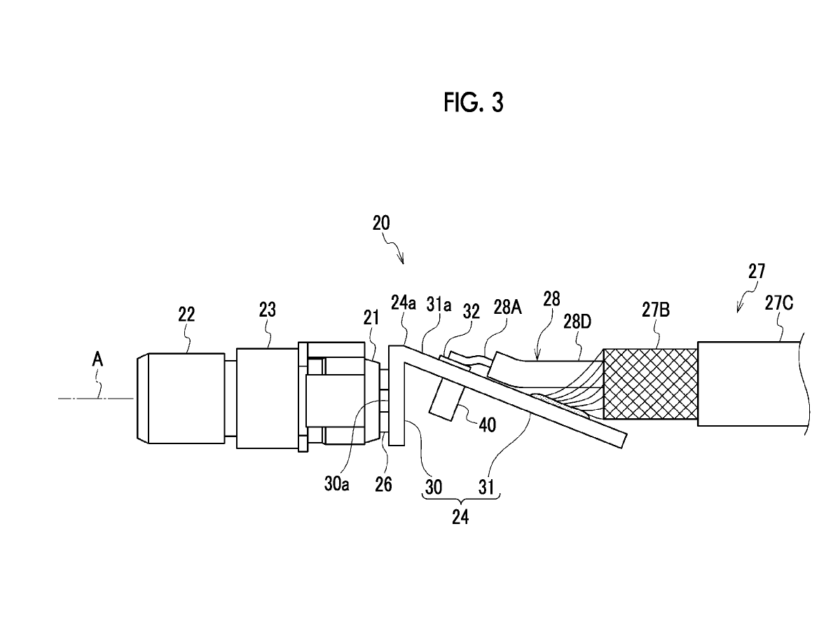

FIGS. 2 and 3 show the structure of a first embodiment of the imaging device that is mounted in the tip portion 10 of the insertion part 6.

An imaging device 20 of the first embodiment includes an image sensor (solid-state imaging element) 21, a lens barrel 22, a sensor holder (holding member) 23, a circuit substrate (substrate) 24 on which the image sensor 21 and the like are mounted, and a transmission cable (composite wire) 27. The image sensor 21 mounted on the circuit substrate 24 is connected to the processor unit 4 through the circuit substrate 24 and the transmission cable 27.

The respective components of the imaging device 20 of the first embodiment will be described below.

The image sensor 21 is a solid-state imaging element, such as a charge coupled device (CCD) image sensor or a complementally metal oxide semiconductor (CMOS) image sensor, and photoelectrically converts an optical image that is formed on the image receiving surface thereof. The image sensor 21 is disposed so that the image receiving surface of the image sensor 21 crosses the longitudinal direction of the insertion part 6. The outer shape of the image sensor 21 is 1 square millimeter or less when the image receiving surface is seen in a normal direction thereto. A plurality of connection terminals 26 to/from which signals or power is input/output are provided on the back of the image sensor 21 opposite to the image receiving surface.

The lens barrel 22 houses the imaging optical system that forms a subject image on the image receiving surface of the image sensor 21.

The sensor holder 23 holds the image sensor 21 on the base end side, and holds the lens barrel 22 on the tip side. Since the sensor holder 23 holds the lens barrel 22 so that the lens barrel 22 is movable along an optical axis A of the imaging optical system, the position of the image sensor 21 relative to the imaging optical system can be adjusted by the movement of the lens barrel 22. The lens barrel 22 is fixed to the sensor holder 23 by, for example, an adhesive or the like after the positioning of the image sensor 21.

The circuit substrate 24 is a flexible substrate on which the image sensor 21 and the like are mounted. The circuit substrate 24 includes a sensor connecting portion (first region portion) 30 and a wire connecting portion (second region portion) 31, and is folded at a boundary 24a between the sensor connecting portion 30 and the wire connecting portion 31.

The sensor connecting portion 30 faces the surface of the image sensor 21 which is opposite to the image receiving surface and on which the connection terminals 26 are provided, and includes a connecting surface 30a that is electrically connected to the connection terminals 26 of the image sensor 21. The wire connecting portion 31 extends from an outer end of the sensor connecting portion 30 toward the base end side of the insertion part 6 and the center of the image receiving surface of the image sensor 21.

The transmission cable 27 is connected to one surface (hereinafter, referred to as a “cable connection surface”) 31a of the wire connecting portion 31 that faces the base end of the insertion part 6. An electronic component 40, such as a capacitor, is mounted on the surface of the wire connecting portion 31 opposite to the cable connection surface 31a.

The circuit substrate 24 is disposed so that the cable connection surface 31a of the wire connecting portion 31 is positioned inside the projected outermost end of the sensor holder 23 in a case in which the circuit substrate 24 is projected with respect to the sensor holder 23 onto a plane orthogonal to the longitudinal direction of the insertion part 6. Further, the sensor holder 23, the image sensor 21, and the circuit substrate 24 are seen from the tip side of the insertion part 6, the circuit substrate 24 is disposed in a region corresponding to the shade of the sensor holder 23 and the image sensor 21.

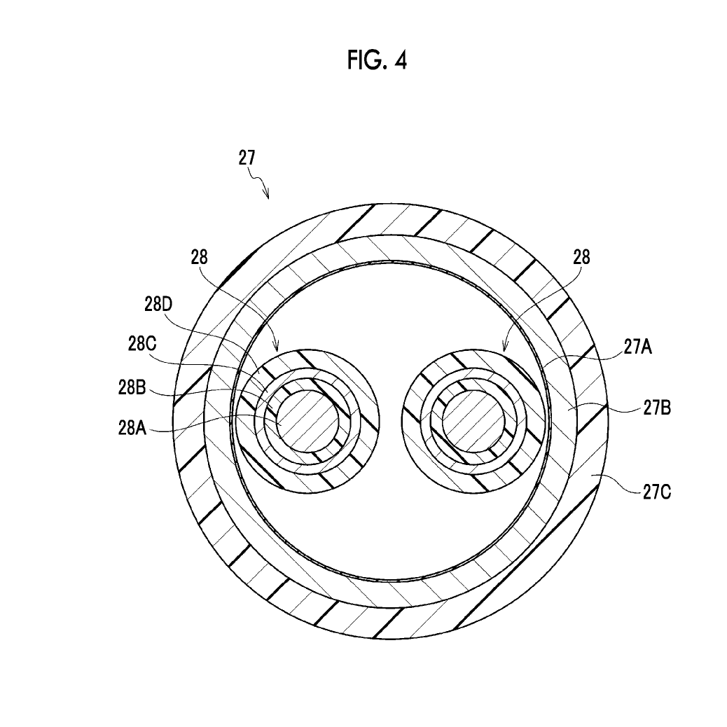

FIG. 4 is a cross-sectional view of the transmission cable 27. As shown in FIG. 4, the transmission cable 27 is a composite wire in which two cables 28 are bundled. The two cables 28 are bundled by a binding layer 27A. A first shield conductor (first external conductor) 27B is provided outside the binding layer 27A. The first shield conductor 27B is covered with a sheath 27C. The first shield conductor 27B is a member that occupies a relatively large volume among members forming the transmission cable 27. Each cable 28 is a shielded wire and includes a core wire 28A, a second shield conductor (second external conductor) 28C that is provided outside the core wire 28A with an insulating layer 28B interposed therebetween, and a sheath 28D that covers the second shield conductor 28C.

As shown in FIGS. 2 and 3, a plurality of lands 32 are formed on the cable connection surface 31a of the circuit substrate 24 and the core wires 28A of the cables 28 are electrically connected to the lands 32, respectively. Further, the first shield conductor 27B of the transmission cable 27 is connected to the cable connection surface 31a by silver paste or solder.

According to the structure of the above-mentioned first embodiment, the first shield conductor 27B, which occupies a relatively large volume among members forming the transmission cable 27, is connected to the cable connection surface 31a of the circuit substrate 24 by silver paste or solder. Accordingly, heat, which is generated from the image sensor 21 and the electronic component 40 mounted on the circuit substrate 24, can be efficiently dissipated to the base end side of the insertion part 6 through the circuit substrate 24 and the first shield conductor 27B. That is, a heat dissipation path, which has a sufficient capacity toward the base end side of the insertion part 6, can be ensured in the imaging device 20.

Further, the circuit substrate 24 is folded at the boundary 24a between the sensor connecting portion 30 and the wire connecting portion 31, and the core wires 28A of the respective cables 28 and the first shield conductor 27B of the transmission cable 27 are connected to the cable connection surface 31a of the wire connecting portion 31 that extends from the outer end of the sensor connecting portion 30 toward the base end side of the insertion part 6 and the center of the image receiving surface of the image sensor 21. Furthermore, the cable connection surface 31a of the circuit substrate 24 is positioned inside the projected outermost end of the sensor holder 23 in a case in which the circuit substrate 24 is projected with respect to the sensor holder 23 onto a plane orthogonal to the longitudinal direction of the insertion part 6. Accordingly, since the core wires 28A and the first shield conductor 27B are connected to the circuit substrate 24, a reduction in the diameter of the imaging device 20 is not inhibited.

Further, since the electronic component 40 is mounted on the surface of the circuit substrate 24 opposite to the cable connection surface 31a, a space in the imaging device 20 can be effectively used as much as possible.

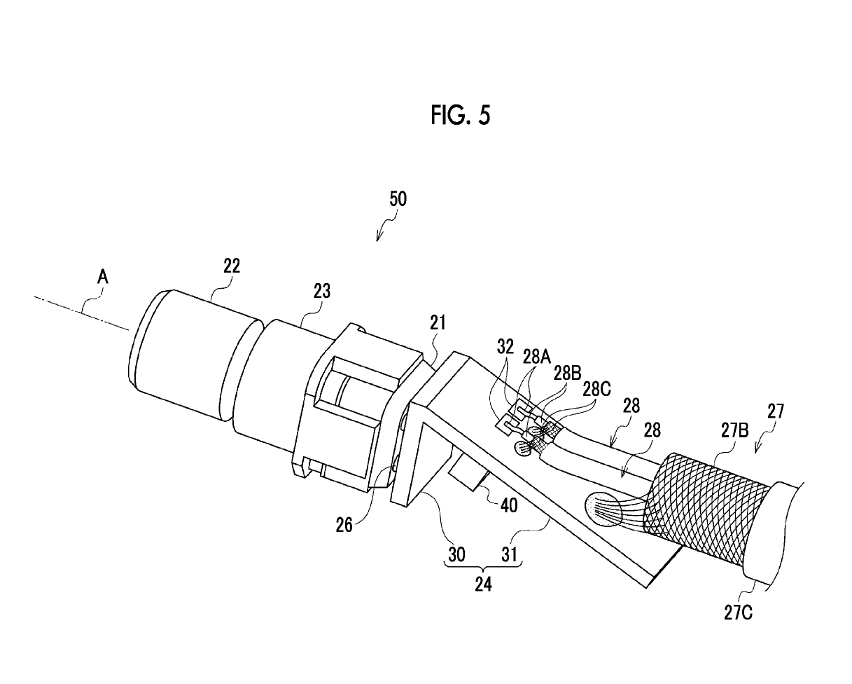

FIG. 5 shows the structure of a second embodiment of the imaging device that is mounted in the tip portion 10 of the insertion part 6. In the following description, the same components as the components having been already described will be denoted by the same reference numerals as the reference numerals having been already described and the description thereof will be appropriately omitted.

An imaging device 50 of the second embodiment includes the image sensor 21, the lens barrel 22, the sensor holder 23, the circuit substrate 24, and the transmission cable 27 as in the imaging device 20 of the first embodiment. In the imaging device 50 of the second embodiment, the respective second shield conductors 28C of two cables 28 are also connected to the cable connection surface 31a of the circuit substrate 24 in addition to the first shield conductor 27B of the transmission cable 27.

According to the structure of the second embodiment, since the second shield conductors 28C are connected to the cable connection surface 31a of the circuit substrate 24, the second shield conductors 28C also function as a heat dissipation path in addition to the first shield conductor 27B. Accordingly, heat, which is generated from the image sensor 21 and the electronic component 40 mounted on the circuit substrate 24, can be more efficiently dissipated than in the first embodiment. That is, a heat dissipation path, which has a more sufficient capacity toward the base end side of the insertion part 6, can be ensured in the imaging device 50.

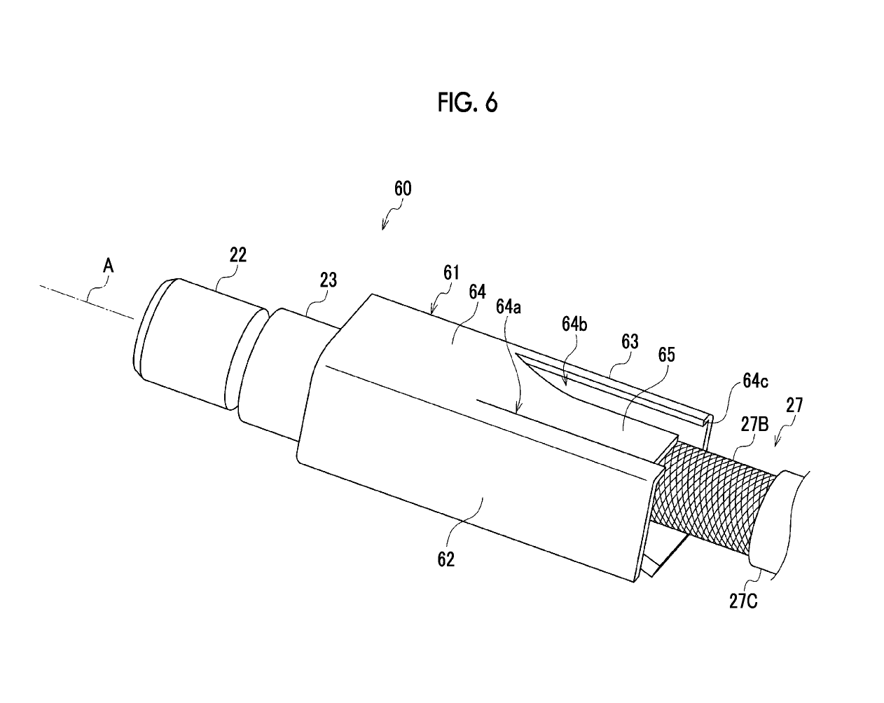

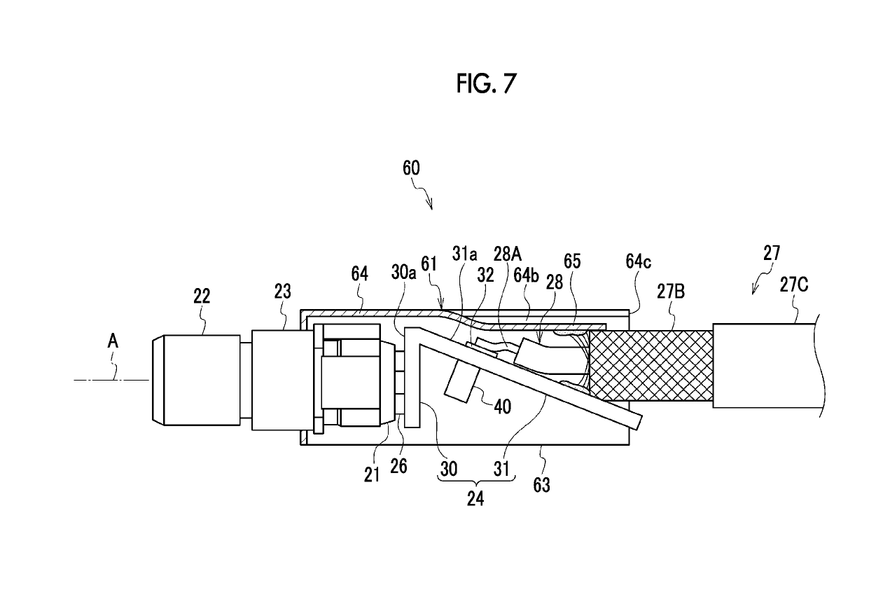

FIGS. 6 and 7 show the structure of a third embodiment of the imaging device that is mounted in the tip portion 10 of the insertion part 6. In the following description, the same components as the components having been already described will be denoted by the same reference numerals as the reference numerals having been already described and the description thereof will be appropriately omitted.

An imaging device 60 of the third embodiment further includes a case 61, which covers the image sensor 21 and the circuit substrate 24 and is made of a conductor, in addition to the image sensor 21, the lens barrel 22, the sensor holder 23, the circuit substrate 24, and the transmission cable 27 of the imaging device 20 of the first embodiment. In the imaging device 60 of the third embodiment, the first shield conductor 27B of the transmission cable 27 is connected to the cable connection surface 31a of the circuit substrate 24 and is also connected to the case 61.

The case 61 includes a pair of side walls 62 and 63 and a ceiling wall 64 that is bridged over the pair of side walls 62 and 63. A pair of slits 64a and 64b is formed parallel to each other at portions of the ceiling wall 64 near the side walls 62 and 63 so as to extend from an end 64c, which is close to the base end side of the insertion part 6, of the ceiling wall 64 to the middle of the ceiling wall 64 in a longitudinal direction. Further, a case element 65, which is a portion of the ceiling wall 64 positioned between both the slits 64a and 64b, is bent toward the inside of the case 61, and the first shield conductor 27B is connected to the case element 65 by silver paste or solder.

According to the structure of the third embodiment, since the first shield conductor 27B of the transmission cable 27 is also connected to the case 61 in addition to the circuit substrate 24, heat, which is generated from the image sensor 21 and the electronic component 40, can be released not only to the first shield conductor 27B but also to the case 61. Accordingly, the heat dissipation performance of the third embodiment can be improved in comparison with that of the first embodiment.

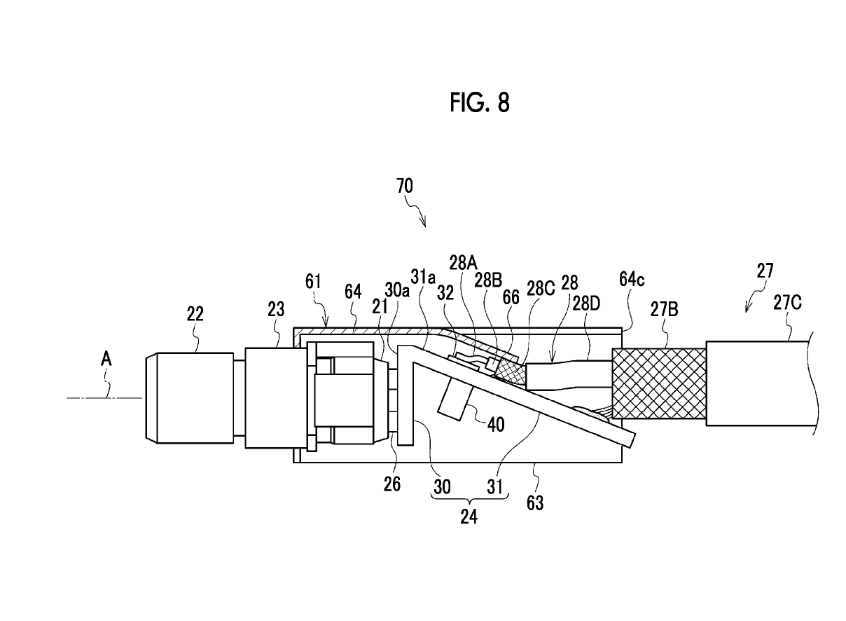

FIG. 8 shows the structure of a fourth embodiment of the imaging device that is mounted in the tip portion 10 of the insertion part 6. In the following description, the same components as the components having been already described will be denoted by the same reference numerals as the reference numerals having been already described and the description thereof will be appropriately omitted.

An imaging device 70 of the fourth embodiment includes the image sensor 21, the lens barrel 22, the sensor holder 23, the circuit substrate 24, the transmission cable 27, and the case 61 as in the imaging device 60 of the third embodiment. In the imaging device 70 of the fourth embodiment, the respective second shield conductors 28C of two cables 28 are also connected to the cable connection surface 31a of the circuit substrate 24 in addition to a first shield conductor 27B of the transmission cable 27 and second shield conductors 28C are also connected to the case 61. Specifically, a case element 66, which is bent toward the inside of the case 61, is formed at the ceiling wall 64 of the case 61 so as to have a structure similar to that of the third embodiment, and the second shield conductor 28C is connected to the case element 66.

According to the structure of the fourth embodiment, since the first shield conductor 27B is connected to the circuit substrate 24 and the second shield conductors 28C are connected to the circuit substrate 24 and the case 61, heat, which is generated from the image sensor 21 and the electronic component 40, can be released not only to the first shield conductor 27B and the second shield conductors 28C but also to the case 61 through the second shield conductors 28C. Accordingly, the heat dissipation performance of the fourth embodiment can be improved in comparison with that of the second embodiment.

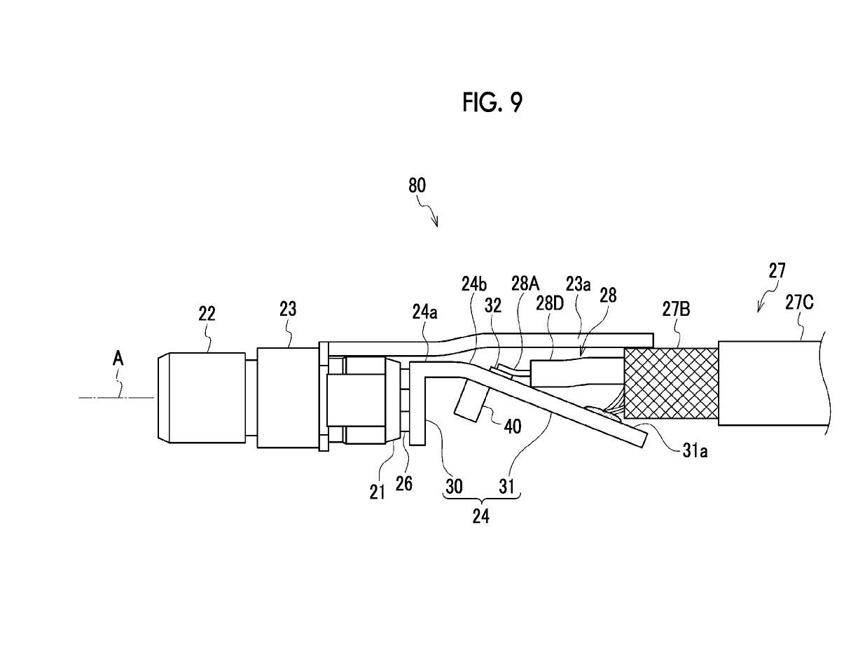

FIG. 9 shows the structure of a fifth embodiment of the imaging device that is mounted in the tip portion 10 of the insertion part 6. In the following description, the same components as the components having been already described will be denoted by the same reference numerals as the reference numerals having been already described and the description thereof will be appropriately omitted.

An imaging device 80 of the fifth embodiment includes the image sensor 21, the lens barrel 22, the sensor holder 23, the circuit substrate 24, and the transmission cable 27 as in the imaging device 20 of the first embodiment. In the imaging device 80 of the fifth embodiment, the first shield conductor 27B of the transmission cable 27 is connected to the cable connection surface 31a of the circuit substrate 24 and is also connected to the sensor holder 23. The sensor holder 23 of this embodiment includes an extending portion 23a that extends toward the base end side of the insertion part 6 from a portion of the sensor holder 23 holding the image sensor 21, and the first shield conductor 27B is connected to the extending portion 23a. Further, the circuit substrate 24 of this embodiment is folded at two positions, that is, a boundary 24a between the sensor connecting portion 30 and the wire connecting portion 31 and a position 24b that is closer to the base end side than the boundary 24a.

According to the structure of the fifth embodiment, since the first shield conductor 27B of the transmission cable 27 is also connected to the sensor holder 23 in addition to the circuit substrate 24, heat, which is generated from the image sensor 21 and the electronic component 40, can be released not only to the first shield conductor 27B but also to the sensor holder 23. Accordingly, the heat dissipation performance of the fifth embodiment can be improved in comparison with that of the first embodiment.

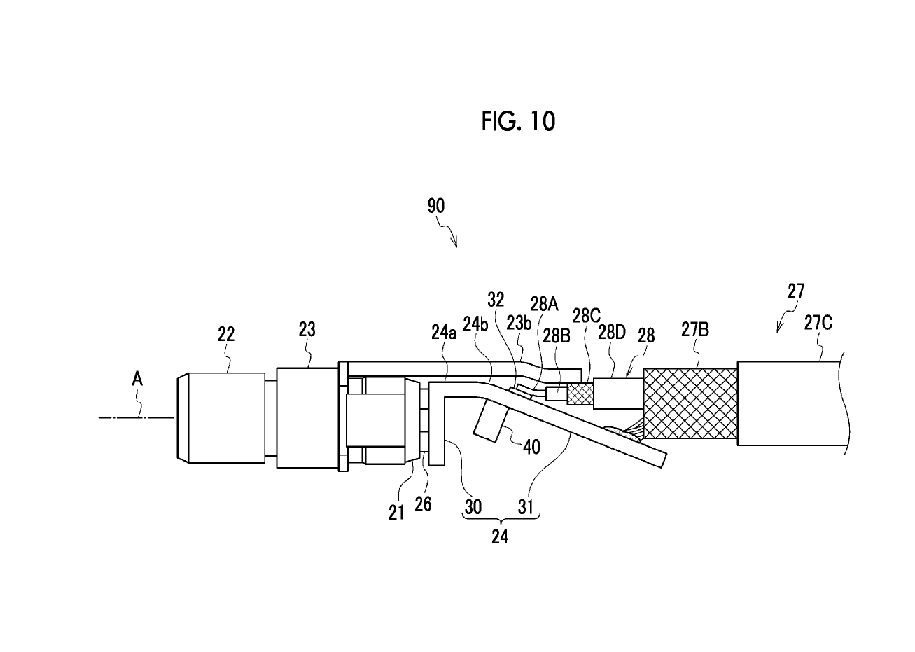

FIG. 10 shows the structure of a sixth embodiment of the imaging device that is mounted in the tip portion 10 of the insertion part 6. In the following description, the same components as the components having been already described will be denoted by the same reference numerals as the reference numerals having been already described and the description thereof will be appropriately omitted.

An imaging device 90 of the sixth embodiment includes the image sensor 21, the lens barrel 22, the sensor holder 23, the circuit substrate 24, and the transmission cable 27 as in the imaging device 20 of the first embodiment. In the imaging device 90 of the sixth embodiment, the first shield conductor 27B of the transmission cable 27 is connected to the cable connection surface 31a of the circuit substrate 24 and the respective second shield conductors 28C of two cables 28 are connected to the sensor holder 23. The sensor holder 23 of this embodiment includes an extending portion 23b that extends toward the base end side of the insertion part 6 from a portion of the sensor holder 23 holding the image sensor 21, and the second shield conductors 28C are connected to the extending portion 23b. Further, the circuit substrate 24 of this embodiment is folded at two positions, that is, a boundary 24a between the sensor connecting portion 30 and the wire connecting portion 31 and a position 24b that is closer to the base end side than the boundary 24a.

According to the structure of the sixth embodiment, since the first shield conductor 27B is connected to the circuit substrate 24 and the second shield conductors 28C are connected to the sensor holder 23, heat, which is generated from the image sensor 21 and the electronic component 40, can be released not only to the first shield conductor 27B but also to the sensor holder 23 through the second shield conductors 28C. Accordingly, the heat dissipation performance of the sixth embodiment can be improved in comparison with that of the first embodiment.

The invention is not limited to the respective embodiments, and can appropriately have modifications, improvements, and the like. For example, the transmission cable 27 of the embodiment has had a structure in which two cables 28 are bundled by the sheath, the shield conductor, and the like, but may have a structure in which three or more cables 28 are bundled. Further, a flexible substrate has been used as the circuit substrate 24 in the embodiments, but a rigid substrate may be used instead of the flexible substrate. Furthermore, structures in which the circuit substrate 24 is folded once or twice have been exemplified in the embodiments, but the circuit substrate may be folded three or more times.

As described above, the endoscope disclosed in this specification is an endoscope including an imaging device that is provided in a tip portion of an insertion part capable of being inserted into a body cavity. The imaging device includes: a solid-state imaging element that is disposed so that an image receiving surface of the solid-state imaging element crosses a longitudinal direction of the insertion part; a substrate that includes a first region portion which faces a surface of the solid-state imaging element on which connection terminals are provided and is connected to the connection terminals and a second region portion which extends from an outer end of the first region portion toward a base end side of the insertion part and a center of the image receiving surface, and is folded once or more; and a composite wire which extends from the base end side of the insertion part toward a tip side of the insertion part and of which a plurality of cables electrically connected to one surface of the second region portion facing a base end of the insertion part are bundled. The composite wire includes a first external conductor provided around the plurality of cables that are bundled, and the first external conductor is connected to a cable connection surface that is one surface of the second region portion to which the plurality of cables are connected.

Further, in a case in which a holding member of holding the solid-state imaging element and the substrate are projected onto a plane orthogonal to the longitudinal direction of the insertion part, the cable connection surface of the second region portion is positioned inside the projected outermost end of the holding member.

Furthermore, each of the cables is a shielded wire, and a second external conductor, which is provided around a core wire of each cable, is connected to the cable connection surface of the second region portion.

Moreover, an electronic component is mounted on a surface of the second region portion opposite to the cable connection surface.

Further, the substrate is a flexible substrate.

Furthermore, the imaging device includes a case that covers the solid-state imaging element and the substrate, and the first external conductor is connected to the case.

Moreover, the imaging device includes a case that covers the solid-state imaging element and the substrate, and the second external conductor is connected to the case.

Further, the imaging device includes a holding member that holds the solid-state imaging element, and the first external conductor is connected to the holding member.

Furthermore, the imaging device includes a holding member that holds the solid-state imaging element, and the second external conductor is connected to the holding member.

### EXPLANATION OF REFERENCES

2: endoscope

6: insertion part

10: tip portion

20, 50, 60, 70, 80, 90: imaging device

21: image sensor (solid-state imaging element)

22: lens barrel

23: sensor holder (holding member)

24: circuit substrate (substrate)

26: connection terminal

27: transmission cable (composite wire)

27A: binding layer

27B: first shield conductor (first external conductor)

27C: sheath

28: cable

28A: core wire

28B: insulating layer

28C: second shield conductor (second external conductor)

28D: sheath

30: sensor connecting portion (first region portion)

31: wire connecting portion (second region portion)

31a: cable connection surface

40: electronic component

61: case

What is claimed is:

1. An endoscope comprising:
an imaging device that is provided in a tip portion of an insertion part capable of being inserted into a body cavity,
wherein the imaging device includes
a solid-state imaging element that is disposed so that an image receiving surface of the solid-state imaging element crosses a longitudinal direction of the insertion part,
a substrate that includes a first region portion which faces a surface of the solid-state imaging element on which connection terminals are provided and is connected to the connection terminals and a second region portion which extends from an outer end of the first region portion toward a base end side of the insertion part and a center of the image receiving surface, and is folded once or more, and
a composite wire which extends from the base end side of the insertion part toward a tip side of the insertion part and of which a plurality of cables electrically connected to one surface of the second region portion facing a base end of the insertion part are bundled,
the composite wire includes a first external conductor provided around the plurality of cables that are bundled, and
the first external conductor is connected to a cable connection surface that is one surface of the second region portion to which the plurality of cables are connected.

2. The endoscope according to claim 1,
wherein in a case in which a holding member holding the solid-state imaging element and the substrate are projected onto a plane orthogonal to the longitudinal direction of the insertion part, the cable connection surface of the second region portion is positioned inside the projected outermost end of the holding member.

3. The endoscope according to claim 1,
wherein each of the cables is a shielded wire, and a second external conductor, which is provided around a core wire of each cable, is connected to the cable connection surface of the second region portion.

4. The endoscope according to claim 2,
wherein each of the cables is a shielded wire, and a second external conductor, which is provided around a core wire of each cable, is connected to the cable connection surface of the second region portion.

5. The endoscope according to claim 1,
wherein an electronic component is mounted on a surface of the second region portion opposite to the cable connection surface.

6. The endoscope according to claim 2,
wherein an electronic component is mounted on a surface of the second region portion opposite to the cable connection surface.

7. The endoscope according to claim 3,
wherein an electronic component is mounted on a surface of the second region portion opposite to the cable connection surface.

8. The endoscope according to claim 4,
wherein an electronic component is mounted on a surface of the second region portion opposite to the cable connection surface.

9. The endoscope according to claim 1,
wherein the substrate is a flexible substrate.

10. The endoscope according to claim 2,
wherein the substrate is a flexible substrate.

11. The endoscope according to claim 3,
wherein the substrate is a flexible substrate.

12. The endoscope according to claim 4,
wherein the substrate is a flexible substrate.

13. The endoscope according to claim 5,
wherein the substrate is a flexible substrate.

14. The endoscope according to claim 6,
wherein the substrate is a flexible substrate.

15. The endoscope according to claim 7,
wherein the substrate is a flexible substrate.

16. The endoscope according to claim 8,
wherein the substrate is a flexible substrate.

17. The endoscope according to claim 1,
wherein the imaging device includes a case that covers the solid-state imaging element and the substrate, and the first external conductor is connected to the case.

18. The endoscope according to claim 3,
wherein the imaging device includes a case that covers the solid-state imaging element and the substrate, and the second external conductor is connected to the case.

19. The endoscope according to claim 1,
wherein the imaging device includes a holding member that holds the solid-state imaging element, and the first external conductor is connected to the holding member.

20. The endoscope according to claim 3,
wherein the imaging device includes a holding member that holds the solid-state imaging element, and the second external conductor is connected to the holding member.
